(19) United States
(12) Patent Application Publication
(10) Pub. No.: US 2025/0367412 A1
(43) Pub. Date: Dec. 4, 2025

(54) MISTAKE-PROOF DEVICE OF CENTRAL VENOUS CATHETERS WITH A GUIDEWIRE

(71) Applicant: CHEN-JUNG PENG, New Taipei City (TW)

(72) Inventor: CHEN-JUNG PENG, New Taipei City (TW)

(21) Appl. No.: 18/731,305

(22) Filed: Jun. 2, 2024

(51) Int. Cl. A61M 25/09

(52) U.S. Cl. CPC A61M 25/09

(57) ABSTRACT

The present invention provides a mistake-proof device of a central venous catheter (CVC) with a guidewire, including: a first fixing portion and a second fixing portion. A first penetrating hole is located at a side of the first fixing portion. One end of the second fixing portion is connected to the first fixing portion. A second penetrating hole is located at one side of the second fixing portion, and the other side of the second fixing portion is rotatably connected to the first fixing portion. The first penetrating hole aligns with the second penetrating hole, and the first fixing portion and the second fixing portion corporately forms an opening to accommodate the CVC. A first stopper is connected to the first fixing portion and extending along a normal direction of the opening, and a stick is configured within the first penetrating hole and the second penetrating hole.

### FIELD OF THE INVENTION

This disclosure is related to the central venous catheters, more particular to a mistake-proof device capable of reducing the occurrence of the guidewire retention.

### BACKGROUND OF THE INVENTION

Guidewire retention is rare but sometimes happened when the operators insert the central venous catheter (CVC) into the patient's vessel. It particularly arises during the catheter placement phase when the operators forget to remove the guidewire from the CVC. Such retention may cause dysrhythmia, vascular injury, thrombosis, embolism, infection, cardiac perforation and tamponade. Generally, the only way to prevent guidewire retention is to instruct the operators to always hold the tip of the guidewire to prevent it from passaging into or out of the vessel.

The commonly used technique for placing CVCs is Seldinger technique. The step causes the guidewire retention most is the step where the catheter is threaded along the guidewire into the vessel. Due to the long length of the CVC, if the operator forgets to confirm that the guidewire is extending from the end of the CVC, the guidewire may inadvertently be retained in the vessel.

A study shows that 32% of the guidewire retention is caused by the operator's mistake. Therefore, there is a need for developing a widget that can help the operators to remember to pull the guidewire out from the CVC.

### SUMMARY OF THE INVENTION

The present invention provides a mistake-proof device of a central venous catheter (CVC) with a guidewire, including: a first fixing portion, wherein a first penetrating hole is located at a side of the first fixing portion; a second fixing portion, wherein one side of the second fixing portion is connected to the first fixing portion, the second fixing portion has a second penetrating hole configured on the other side, the second penetrating hole aligns with the first penetrating hole, and the first fixing portion and the second fixing portion corporately forms an opening to accommodate the CVC; a stick configured within the first penetrating hole and the second penetrating hole; a first stopper extending from the first fixing portion along a normal direction of the opening.

Preferably, a pivot is configured between the first fixing portion and the second fixing portion.

Preferably, the stick is sunken into one end of the second penetrating hole.

Preferably, a diameter of the second penetrating hole is greater than a diameter of the guidewire

Preferably, a length of the first stopper is less than a length of a catheter of the CVC and great than half of the length of the catheter of the CVC.

Preferably, the second fixing portion is configured with a second stopper extending along the normal direction of the opening, and a catheter of the CVC is located in between the first stopper and the second stopper.

Preferably, at least one portion of the first stopper and the second stopper are in an arc shape, and the first stopper and the second stopper corporately surround the catheter.

In summary, practically, when the mistake-proof device is fixed onto the CVC and the operators tend to insert the CVC into the vessel, the catheter will be blocked by the stoppers, the operator is not able to fully insert the catheter into the vessel without removing the mistake-proof device, so the operators must remove the mistake-proof device before keeping inserting the catheter into the vessel. Particularly, the operators can use the guidewire to push the stick out of the penetrating hole when operating, such that the mistake-proof device may help the operators to remember to pull the guidewire out from the end of the CVC. Thus, the occurrence of the guidewire retention may be reduced.

### BRIEF DESCRIPTION OF THE DRAWINGS

FIG. 1 is a schematic diagram of the mistake-proof device of a CVC in one embodiment of the present disclosure.

FIG. 2 is a schematic diagram of the mistake-proof device fixing onto the CVC in one embodiment of the present disclosure.

FIGS. 3a and 3b are a schematic diagram of the mistake-proof device of a CVC in one embodiment of the present disclosure.

FIGS. 4a and 4b are a schematic diagram illustrating the first fixing portion and the second fixing portion are integrated into one element in one embodiment of the present disclosure.

FIG. 5 is a schematic diagram of the mistake-proof device of a CVC in one embodiment of the present disclosure.

### DETAILED DESCRIPTION OF THE INVENTION

In order to make the aforementioned and/or other purposes, benefits, and features of the present disclosure clearer and more understandable, the following detailed description is provided, using preferred embodiments as examples.

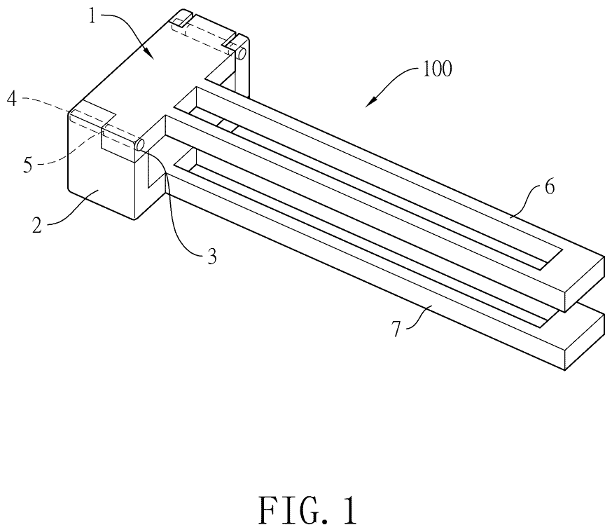

As shown in FIG. 1, a mistake-proof device 100 includes a first fixing portion 1 and a second fixing portion 2. The first fixing portion 1 has a first penetrating hole 3 located at a side of the first fixing portion 1. The other side of the first fixing portion 1 is connected to the second fixing portion 2. For example, a pivot is configured between the first fixing portion 1 and the second fixing portion 2, such that the fixing portion 1 may rotate relative to the second fixing portion 2. The second fixing portion 2 has a second penetrating hole 4 configured on one side. When the first penetrating hole 3 is aligned with the second penetrating hole 4, the first fixing portion 1 and the second fixing portion 2 can form a ring-shape structure having an opening where a central venous catheter (CVC) can be placed within.

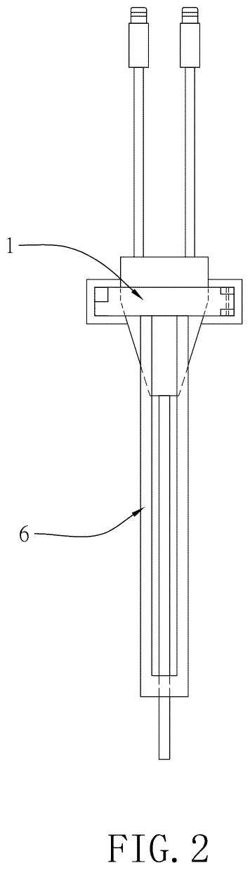

A stick 5 penetrates through the first penetrating hole 3 and the second penetrating hole 4 when the first penetrating hole 3 and second penetrating hole 4 align with each other. Such that, as shown in FIG. 2, the first fixing portion 1 and the second fixing portion can be fixed together, and the CVC can be fixed within the opening.

In one embodiment, a diameter of either one of the first penetrating hole 3 or the second penetrating hole 4 is equal to a diameter of the stick 5. Therefore, the stick 5 can be tightly inserted into the first penetrating hole 3 or the second penetrating hole 4. In another embodiment, the diameter of the second penetrating hole 4 is greater than a diameter of the guidewire of the CVC, so that the operators can use the guidewire to push the stick 5 out of the first penetrating hole 3 and the second penetrating hole 4.

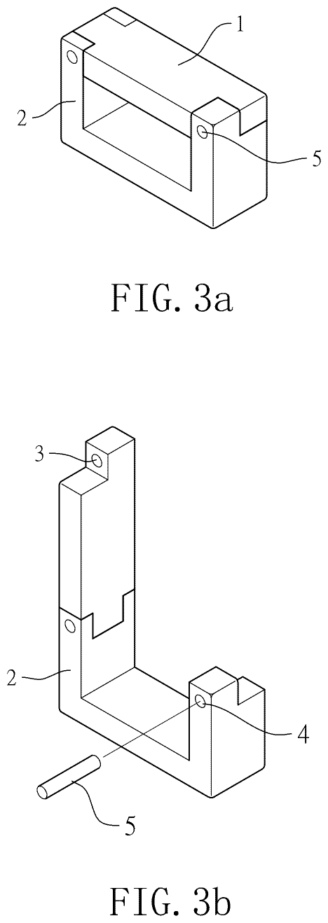

As shown in FIGS. 3a and 3b, when using the mistake-proof device 100, the operators can use the guidewire to push the stick 5 out of the first penetrating hole 3 and the second penetrating hole 4 to open the mistake-proof device 100. Therefore, in the practical scenario, the mistake-proof device 100 may be a reminder that helps the operators to remember to pull the guidewire out from the CVC after inserting the tip of the catheter into the vessel and use the guidewire to open the mistake-proof device in order to continue inserting the catheter. Such that the mistake-proof device can effectively reduce the occurrence of the guidewire retention.

In another embodiment, when the stick 5 is placed into the first penetrating hole 3 and the second penetrating hole 4, the stick 5 may not align with the first penetrating hole 3 and the second penetrating hole 4, with one end of the stick 5 is sunken into one end of the second penetrating hole 4. Therefore, when the operators intend to use the guidewire to open the mistake-proof device 100, the guidewire can easily be inserted into the second penetrating hole 4, with the diameter of the second penetrating hole 4 greater than the diameter of the guidewire, to push the stick 5 out of the first penetrating hole 3 and the second penetrating hole 4.

It should be noted that the stick 5 can be pushed out of the first penetrating hole 3 and the second penetrating hole 4 by any element with the diameter less the second penetrating hole 4, and the present disclosure is not limited thereto.

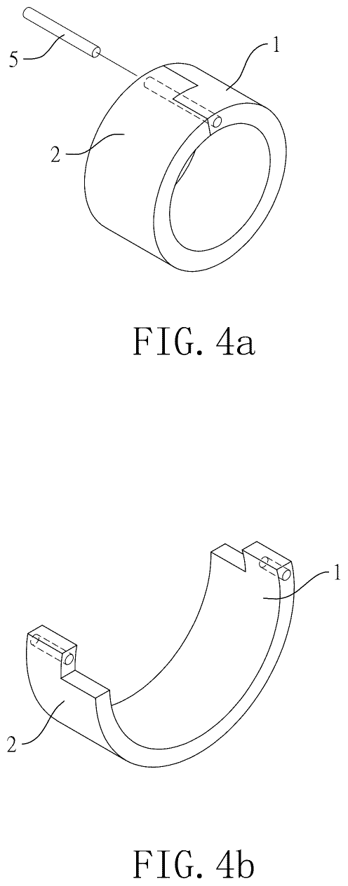

In another embodiment, as shown in FIGS. 4a and 4b, the first fixing portion 1 and the second fixing portion 2 can be integrated into one element with one end of the first fixing portion 1 fixing to one end of the second fixing portion 2 and form a ring shape structure to accommodate the CVC. The first fixing portion 1 and the second fixing portion 2 are made of elastic material, such as plastic. Therefore, the first fixing portion 1 and the second fixing portion 2 are movable relative to each other. When one end of the first fixing portion 1 is spaced apart from the second fixing portion 2, the first fixing portion 1 and the second fixing portion 2 may return to the original position by the restoring force of the elastic material. In practical, the mistake-proof device 100 in this embodiment may be fixed onto or removed from the CVC rapidly and easily when using. It should be noted that, in this embodiment, the first fixing portion 1 and the second fixing portion 2 are still equipped with the stoppers, and the function of the stops is similar to the previous embodiments, therefore the details regarding the stoppers will not be further described herein.

The first fixing portion 1 further includes a first stopper 6. The first stopper 6 is located at the middle portion of the first portion 1 and extending along a normal direction of the opening. When inserting the CVC into the vessel, the operators need to open the mistake-proof device 100 after inserting the tip of the vessel into the vessel, otherwise the first stopper 6 may stop the operators from keeping inserting the catheter into the vessel. In one embodiment, the second fixing portion may have a second stopper 7. The second stopper 7 is configured on the middle portion of the second stopper 7 and extending along the normal direction of the opening. The first stopper and the second stopper 7 corporately sandwich the catheter in between to ensure the stoppers can effectively stop the operators from keeping inserting the catheter into the vessel.

In one embodiment, a length of the first stopper 6 and a length of the second stopper 7 are less than a length of the catheter of the CVC, but, preferably, longer than half the length of the catheter part of the CVC. Such that the effect of preventing guidewire retention can be even improved.

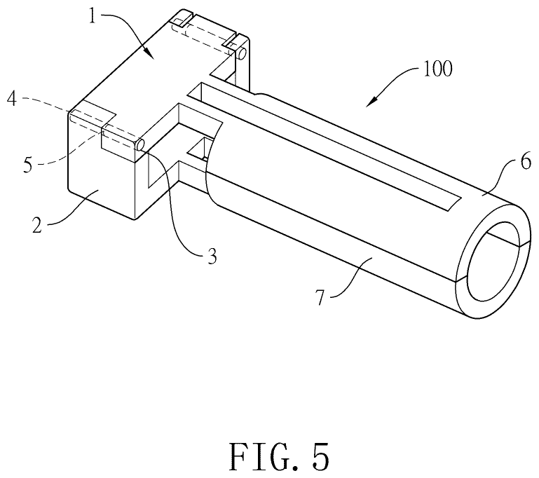

In another embodiment, as shown in FIG. 5, at least one portion of the first stopper 6 and the second stopper 7 can be in an arc shape. The first stopper 6 and the second stopper 7 may fully surround the catheter part of the CVC when the mistake-proof device 100 is fixed onto the CVC. In practical, the catheter part of the CVC is soft and flexible, the arc-shaped stoppers may ensure the catheter is completely located within the first stopper 6 and the second stopper 7 to ensure the stoppers are able to stop the operators from keeping inserting the catheter.

Preferably, holes may be configured on the first stopper 6 and the second stopper 7, such that the operators may be able to inspect whether there is any defect on the catheter before inserting the catheter into the vessel.

In summary, practically, when the mistake-proof device is fixed onto the CVC and the operators tend to insert the CVC into patient's vessel, the catheter will be blocked by the stoppers, the operators will not be able to fully insert the catheter into the vessel without removing the mistake-proof device, so the operators must remove the mistake-proof device to keep inserting the catheter into the vessel. Particularly, the operators can use the guidewire to push the stick out of the penetrating hole when operating, such that the mistake-proof device may help the operators to remember to pull the guidewire out from the end of the CVC. Thus, the occurrence of the guidewire retention may be reduced.

The above description and explanation are only descriptions of the preferred embodiments of the present invention. Those with ordinary knowledge of this technology can make other modifications based on the patent scope defined below and the foregoing description. However, these modifications should be still regarded as the creative spirit of the present invention and be within the scope of the rights of the present invention.

What is claimed is:

1. A mistake-proof device of a central venous catheter (CVC) with a guidewire, comprising:
a first fixing portion, wherein a first penetrating hole is located at one side of the first fixing portion;
a second fixing portion, wherein a second penetrating hole is located at one side of the second fixing portion, and the other side of the second fixing portion is connected to the first fixing portion, the second penetrating hole aligns with the first penetrating hole, and the first fixing portion and the second fixing portion corporately forms an opening to accommodate the CVC;
a stick configured within the first penetrating hole and the second penetrating hole; and
a first stopper extending from the first fixing portion along a normal direction of the opening.

2. The mistake-proof device of a CVC with a guidewire according to claim 1, wherein a pivot is configured between the first fixing portion and the second fixing portion.

3. The mistake-proof device of a CVC with a guidewire according to claim 2, wherein the stick is sunken into one end of the second penetrating hole.

4. The mistake-proof device of a CVC with a guidewire according to claim 3, wherein a diameter of the second penetrating hole is greater than a diameter of the guidewire.

5. The mistake-proof device of a CVC with a guidewire according to claim 1, wherein a length of the first stopper is less than a length of a catheter of the CVC and great than half of the length of the catheter of the CVC.

6. The mistake-proof device of a catheter with a guidewire according to claim 1, wherein the second fixing portion is configured with a second stopper extending along the normal direction of the opening, and a catheter of the CVC is located in between the first stopper and the second stopper.

7. The mistake-proof device of a catheter with a guidewire according to claim 6, wherein at least one portion of the first stopper and the second stopper are in an arc shape, and the first stopper and the second stopper corporately surround the catheter.
